(19) United States
(12) Patent Application Publication
(10) Pub. No.: US 2004/0225214 A1
(43) Pub. Date: Nov. 11, 2004

(54) Method of high-throughput quantitative pet for small animal imaging using a large-bore pet scanner for the assessment of pet tracer performance

(75) Inventors: Dinko Eduardo Gonzalez Trotter, Clifton Park, NY (US); Ravindra Mohan Manjeshwar, Guilderland, NY (US)

Correspondence Address: FOLEY AND LARDNER, SUITE 500, 3000 K STREET NW, WASHINGTON, DC, 20007, US

(73) Assignee: GENERAL ELECTRIC COMPANY

(21) Appl. No.: 10/430,429

(22) Filed: May 7, 2003

(51) Int. Cl. A61B 6/00

(52) U.S. Cl. 600/436

(57) ABSTRACT

A method of tracer evaluation using PET (positron emission tomography), includes introducing the tracer into a small animal; scanning the animal in a large-bore PET scanner; and quantitating a concentration of tracer in a predetermined portion of the animal.

### BACKGROUND OF THE INVENTION

The present invention relates generally to a non-invasive in vivo method of examining small bodies such as the organs of small animals, and more specifically to a non-invasive method of examining small bodies in a large-bore clinical PET scanner in a manner that enables quantitative examination of PET tracer distributions in the small animals/bodies.

Antibody fragments can show very high affinity and specificity for antigens (receptors) that are commonly over-expressed in human cancers. Antibody fragments labeled with an appropriate PET (Positron Emission Tomography) radioisotope may provide an effective means of detection of very small lesions by virtue of high target-to-background ratios coupled with adequate tumor activity concentration. By targeting the appropriate antigen, antibody PET imaging may also prove to be very effective in the assessment of tumor response to therapy, since in principle the intensity of a distribution in a PET image of a treated tumor will be proportional to the number of antigens present and to the number of tumor cells that have not been destroyed by therapy. This is in contrast to FDG (F-18 fluorodeoxygluclose) PET imaging, where the tumor signal may actually increase for some time after treatment due to inflammation and/or modified tumor cell metabolism of FDG in response to stress. Furthermore, antibody PET may provide superior detection and response assessment of lesions that happen to be inside or near organs that normally show relatively high concentrations of FDG, such as the brain, heart, bone marrow (after chemotherapy treatment), liver and bladder.

The assessment of PET tracer/drug performance is therefore vital in the development of diagnostic/therapeutic pharmaceuticals. Currently biodistribution studies of compounds are typically conducted ex-vivo by injecting a large number of animals (such as murine animals) with a radioactive compound/drug/tracer and sacrificing animal cohorts at various time-points.

It should be noted that throughout this disclosure the term “small animal” will be taken to mean, but not be specifically limited to, murine animals such as mice and other relatively small animals that are available for research purposes.

For example, the testing of different PET tracers inevitably involves the use of a large number of small murine animals (mice) wherein each of these small animals is dosed with a predetermined amount of a PET tracer. After a given amount of time has passed the mice (as they will be referred to for the sake of simplicity) are individually euthanized and subjected to a necropathic examination wherein each animal is autopsied. Selected organs/tissue is removed and the amount of tracer which is contained therein, measured.

In the case of different tracers, it is necessary to repeat each test a number of times for each tracer and for each variant such as concentration, time or the like, that is deemed important to the study. This method therefore suffers from the drawbacks that it is very time consuming in that that each of a large number of animals must be euthanized and each animal then carefully dissected in manner which is consistent with the particular tests which are being conducted.

It further suffers from the drawback that the euthanization of each animal means that the same animal cannot be relied upon to provide further information. That is to say, to determine the amount of tracer which has accumulated in a given organ or tissue at each of 1, 3, 5 and 10 hour intervals (for example) following the initial tracer dosage, it is necessary to repeat the procedure with a series different animal or animals (mice) wherein the amount of time between the introduction of the tracer and the euthanization is extended, merely by way of example, from 3 to 5 hours and so on. Apart from being very labor intensive, this time consuming procedure introduces “noise” into the results in that each animal is not exactly the same and there is inevitably an animal-to-animal variation in biological activity and physiology.

### SUMMARY OF THE INVENTION

A first aspect of the invention resides in method comprising image point tracer evaluation using PET (positron emission tomography) in small animals using a large bore PET scanner.

A second aspect of the invention resides in a method of tracer evaluation using PET(positron emission tomography), comprising: introducing the tracer into a small animal; scanning the animal in a large-bore PET scanner; and estimating a concentration distribution of the tracer in a predetermined portion of the animal.

A third aspect of the invention resides in a method of evaluating tracers in a small animals comprising the steps of: introducing the tracer into a plurality of small animals; simultaneously scanning the plurality of small animals in a large-bore PET scanner; and estimating a concentration distribution of tracer in the plurality of small animals.s method of measuring pharmacological agent distribution at different time points, comprising: labeling the pharmacological agent with a tracer; dosing a plurality of small animals with the labeled pharmacological agent; placing the small animals in a large-bore PET (positron emission tomography) scanner; scanning the small animals; and determining a concentration distribution of the tracer in the small animals.

A fifth aspect of the present invention resides in a non-invasive method comprising: quantitative imaging in a large-bore scanner for PET (positron emission tomography) tracer/drug assessment in small animals.

A sixth aspect of the invention resides in a method of operating a PET (positron emission tomography) scanner to account for loss of events and quantitative inaccuracies in PET imaging, comprising: modeling a distribution of positron-electron annihilation events by: convolving a first distribution of positron-emitting radionuclides using a first predetermined kernel to produced a second distribution result; masking the second distribution result with a three-dimensional map of a physical medium where the distribution of radionuclides is located so as to suppress positron-electron annihilation events from a low-density medium and to produce a third distribution result; and convolving the third distribution result with a kernel representative of the measurement system to produce a fourth distribution result which takes into account the loss of positron-electron annihilation events.

### BRIEF DESCRIPTION OF THE DRAWINGS

The various aspects and features of the invention will become more clearly appreciated from the following description taken with the appended drawings wherein:

FIG. 1 is a perspective view showing a phantom which includes spherical inserts containing PET tracer material immersed in a predetermined medium and which was used in connection with the development of the correction processes which are applied in accordance with the embodiments of the invention.

FIG. 2 is a coronal PET image derived using the phantom depicted in FIG. 1 with 1/20 background-to-target activity concentration ratios.

FIG. 3 is a graph showing a sinogram slice going through the phantom (1:10 background-to-target activity concentration) and wherein the modest contribution of scatter and prompt gamma events is evident by the small tails and low background counts outside of the physical boundaries of the phantom.

FIG. 4 is a graph showing profiles across an I-124 line source fitted by a Gaussian.

FIG. 5A-FIG. 5F are graphical plots which show maximum measured activity concentration and recovered activity concentration as a function of sphere volume for various background-to-target ratios and distances from the z-axis of the scanner. The horizontal line shows the ground-truth activity concentration in the spheres.

FIG. 6 is a perspective view of an anesthetized mouse disposed in a large bore PET scanner.

FIG. 7 is a 48 hr post-injection anti I124 HER2/neu PET image of the mouse shown in FIG. 6.

FIG. 8 shows from left to right: a simulated spherical distribution of activity which was convolved by a positron-range kernel, then masked by the medium with density above that of air, convolved again by a system kernel resulting in the estimated PET distribution taking into account the positrons loss in air.

FIG. 9 is a perspective view showing a PET scanner and associated hardware including a memory in which the scan data can be stored along with the resulting process data which is implemented in accordance with the embodiments of the invention.

### DETAILED DESCRIPTION OF THE PREFERRED EMBODIMENTS

The aspects of the invention relate to a non-invasive PET method of measuring pharmacological agent (e.g. antibody) distributions, wherein the agents have been labeled with a PET tracer. It should be noted at this time that the embodiments of the invention are not limited to antibody distribution and that the tracers to which the embodiments of the invention are directed can also be attached to a variety of different elements such as ligands, peptides, small molecules, etc., thus enabling a wide range of pharmacological research to be carried out.

The measurements can be carried out at different time-points in small animals, allowing for the use of fewer animals per study and smaller quantities of pharmacological agent (e.g. antibody) that may be expensive/difficult to produce. This non-invasive PET method increases the rate at which tests can be carried out and enables screening of the performance of various radiotracers before carrying out a detailed necropsy study.

PET analysis/assay techniques enable non-invasive biodistribution studies by imaging the same group of live animals at a plurality of different time-points and enables assessing the concentration of radiolabeled agent (e.g. antibody) in different tissues in a manner that interferes minimally with an animal's normal biology. This increases the throughput of the examination process

This task imposes on PET the quantitative requirement of determining the absolute concentration of radioisotope within small objects, such as xenograft tumors and internal organs of mice, which may be a fraction of a cubic centimeter in size. This presents a challenge for traditional clinical PET systems whose intrinsic resolution is ˜5 mm FWHM (Full Width Half Maximum) at best, resulting in severe blurring of small activity distributions and systematic errors in any direct measurements of concentration attempted from the PET image.

On the other hand, a clinical large-bore PET scanner which has a bore of 50 cm or more and which is adapted for the examination of adult humans, provides sufficient space to allow for the simultaneous scanning of several animals by virtue of its large field of view. In addition, its data acquisition properties are well understood, and its collimators provide an effective means of rejecting scatter and spurious gamma coincidences that are associated with the prompt gamma emissions of many long-lived PET radioisotopes that are suitable for antibody imaging.

Quantitative accuracy was established for a model that determines the activity concentration measurements of tracer distributions that resemble those from murine models with xenograft tumors and imaged with tracer labeled diabodies using a clinical PET/CT scanner, to produce in vivo longitudinal biodistribution data of tracer diabody PET tracers.

This enables the acquisition of quantitative biodistribution data from PET (i.e. in vivo) from multiple small animals scanned in a large-bore scanner. With this, time-dependent behavior of a PET tracer/drug can be followed over time for multiple animals without having to sacrifice them at every time point of the study.

In accordance with a first aspect of the invention, multiple small animals that have been injected with a PET tracer are positioned within the field of view of a large-bore PET scanner. PET data are acquired and reconstructed. Biodistribution data is then extracted through PET organ/tumor/tissue quantation.

The proposed methodology enables the in vivo quantitative assessment of PET-labeled compounds at multiple time-points for multiple animals without having to sacrifice them, thus increasing the PET-labeled compound evaluation throughput and accuracy.

In order to achieve the above in vivo quantitative assessment of PET-labeled compound it is necessary to develop a suitable method of processing the data which is obtained from the large-bore PET scanner. This includes the development of recovery coefficients in accordance with a second aspect of the invention. In order to avoid repetitive calculation of recovery coefficients, it is within this scope of the invention to pre-calculate plurality of recovery coefficients for a range of different parameters and storing the plurality of recovery coefficients in a look up table. These stored recovery coefficients can then be obtained from the look up table based by interpolation based on the predetermined parameters.

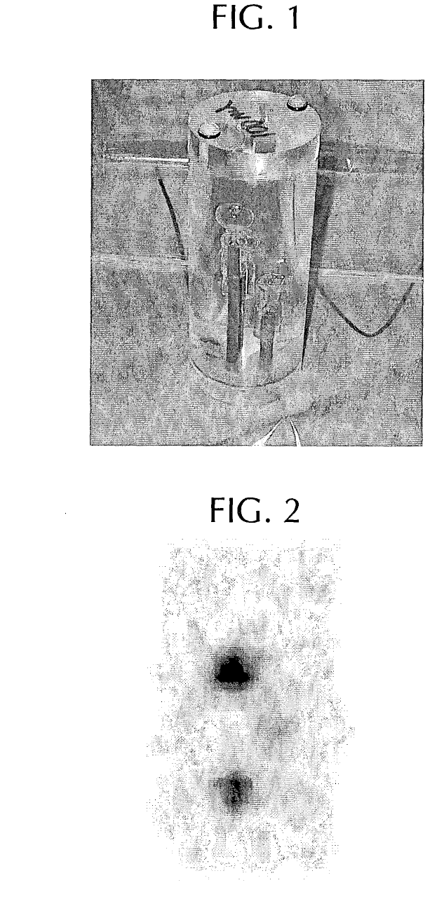

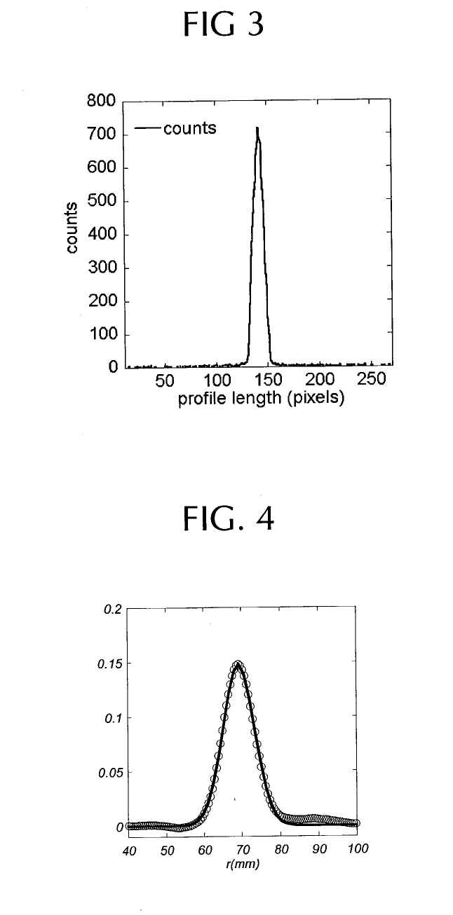

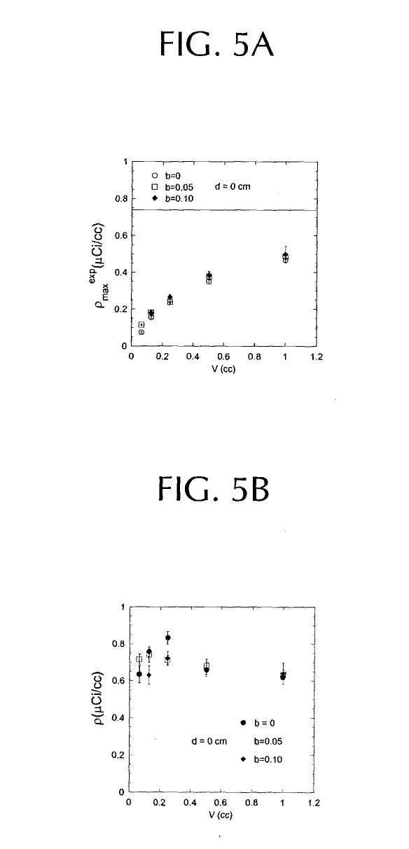

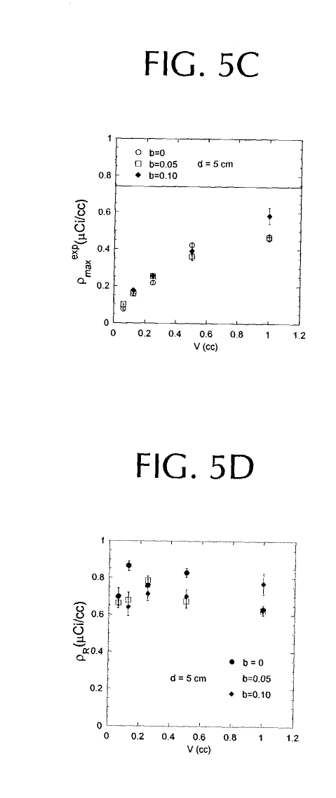

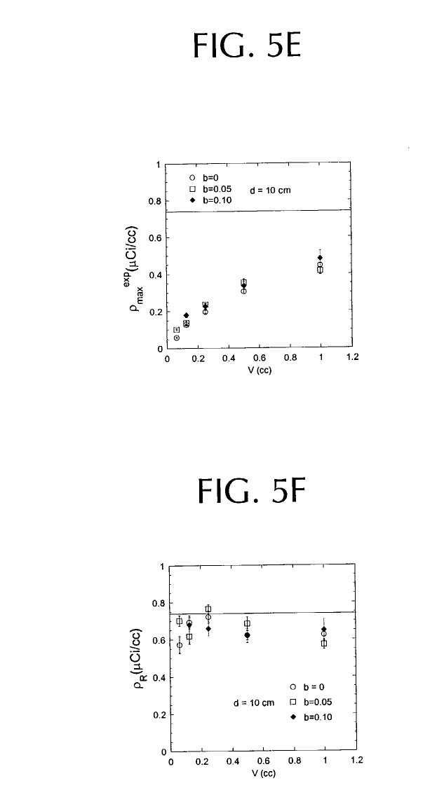

In order to develop the recovery coefficients, a mini-phantom (see FIG. 1) which includes spherical inserts in attenuating media (emulating small-animals) was imaged in a clinical PET scanner. The phantom was imaged with the spherical inserts over a range of diameters, activity distributions, source-to-background ratios and PET radioisotopes at various positions within the bore of the scanner. Data acquired from the scanner were reconstructed. For a given reconstruction algorithm, a look-up table of spatial correction coefficients was obtained for distributions of certain geometry (e.g. spheres of different sizes) and various sphere-to-background activity concentrations and different PET isotopes (e.g. F-18, I-124).

When mice were scanned, the appropriate recovery coefficients were selected according to organ/tumor/tissue size, shape, location and source-to-background ratio. These were stored as N-dimensional recovery coefficients in a look-up-table where the N-dimensions are the N parameters (e.g. geometry, source-to-background ratio, radio-isotope, reconstruction method etc.).

The selected recovery coefficient was then applied to the in-vivo PET image on a pixel-by-pixel basis in order to obtain data that is equivalent to a biodistribution numbers (e.g. percent injected dose per gram of tumor/organ/tissue), or to a single pixel in the image belonging to a particular tumor/organ/tissue to obtain the a biodistribution number for that particular tumor/organ/tissue

A third aspect of the invention resides in a process which involves the above mentioned correction coefficients to further improve the accuracy of the assay. In accordance with this third aspect of the invention, the distribution of positron-electron annihilation events is modeled by a convolution of the distribution of positron-emitting radionuclides using an appropriate kernels.

The resulting distribution of annihilation events is masked by a three-dimensional map of the physical medium where the distribution of radionuclides is located. Thus, the annihilation events that could not have physically occurred because of low-density medium (such as air) surrounding the distribution are suppressed.

The resulting distribution is convolved once again with a kernel representative of the measurement system (i.e. PET scanner). Thus a distribution is obtained which takes into account the loss of positron events. Appropriate resolution recovery factors are obtained by comparison of original distribution of radionuclide with the one resulting from the convolution and masking operations—thus enabling the calculation of activity concentrations from in vivo data. The loss of positron events due to the finite size of the attenuating medium can also be estimated and used for correction of measured activities.

This third aspect of the invention therefore overcomes the problem wherein positron emission tomography (PET) relies on the detection of the annihilation of positrons emitted from a radionuclide (e.g. F-18, I-124, Ga-68) with electrons present in the surrounding medium. Under normal circumstances the standard assumption is that activity distribution of interest is embedded in tissue of more-or-less homogeneous density (typically that of water). Positrons are emitted from the radionuclide with a certain spectrum of energies, which determine the distribution of distances that a positron may travel through surrounding medium before annihilating with an electron.

In some situations, however, the distribution radionuclide may be in close enough proximity to a medium of density significantly different from that of water (for example, air). In such cases, some positrons have a significant probability of reaching air, and hence their probability of annihilating in close proximity to their point of emission is drastically reduced and they can be considered as “lost” to measurement. This situation is possible for small amounts (on the order of a gram or less) of tissues whose activity (based on a PET radionuclide) is measured in test tubes, and for xenograft tumors embedded under the skin of animals.

This effect has actually been observed for PET radionuclides with relatively high-energy positron-emission spectra, such as I-124. Therefore, a method of properly accounting for the loss of positron annihilation events is necessary in order to improve the accuracy of estimates of PET radionuclide activity and activity concentrations.

In accordance with a first aspect of the invention, in vivo time-dependent PET imaging is used as a tool in the assessment of radiotracer performance in murine models. The PET radionuclide I-124 was used during the development of this first aspect of the invention because of its attractive labeling properties and decay half-life (4.2 days) suitable for imaging tumors using antibody fragments.

I-124 is a PET radionuclide suitable for antibody imaging because of its favorable binding chemistry, long half-life (t_{1/2}=4.2 days) and rapid clearance from most tissues. On the other hand, I-124 has a positron emission probability per disintegration of only 23%, and a gamma-ray emission probability of over 90% per disintegration. A significant number of prompt gammas are emitted in cascade with positrons, which produce true photon coincidence detections that are spatially uncorrelated, resulting in a quasi-uniform background in the emission sinogram. The mean positron energy of I-124 (819 keV) is significantly greater than that of F-18 (250 keV), which is expected to result in additional resolution degradation of the PET image.

Nevertheless, it should be noted that aspect of the invention are in no way limited to the use of I-124 and that it is merely exemplary of the type of tracer that can used/tested.

Experiments were conducted to provide a quantitative analysis of I-124 PET images on a GE Discovery LS system using a small-animal phantom to obtain recovery coefficients (second aspect of the invention), which were then applied to in vivo murine PET data.

The PET-obtained quantitation of tumor % injected dose/gram compared favorably with those obtained from a conventional biodistribution study done ex-vivo on the same mice.

The small-animal phantom (see FIG. 1) consisted of a 4×8 cm water-filled acrylic cylinder with hollow spheres also filled with water and ranging in volume from 0.0625 ml to 1.0 ml. The phantom was placed in a PET scanner of the nature depicted in FIG. 9.

The I-124 activity concentration in the spheres was 0.74±0.05 microCi/cc and the background activity concentrations were varied from 0 to 1/20 to 1/10 of the spheres. Activity concentration data were acquired at 0 cm, 5 cm and 10 cm from the longitudinal axis of the scanner. FBP (filtered backprojection) images were reconstructed with a 2 mm FWHM Gaussian post-filter. The background activity concentration was estimated from regions of interest (ROIs) near the center of phantom and a few cm from the spheres. Recovery coefficients were theoretically calculated for spheres of different volume, sphere-to-background concentrations and distance from the scanner's longitudinal axis. The recovery coefficients were applied to the maximum sphere activity concentration measured from the PET images, thus obtaining a “recovered” activity concentration to be compared with the known activity concentration of the spheres.

Background activity concentrations were measured from the PET images with a bias of ˜4 % and a standard deviation of ˜5 %. For all the spheres the overall recovered activity concentration bias and standard deviation was −9 % and 10%, respectively. I-124 diabody PET images of three mice with tumor xenografts were then analyzed using the techniques described above. Tumor percent injected dose per gram (viz., % ID/g=(decay-corrected activity concentration in the tumor divided by the injected dose) estimated from the PET images (for example 0.379, 1.73, 4.77) were comparable to those obtained from biodistribution studies (i.e. 0.362, 1.92, 5.13). The ability to perform quantitative imaging on murine I-124 antibody fragment PET images using a large-bore clinical scanner enables high-throughput studies to evaluate the performance of PET tracers in a timely and cost-effective manner by imaging multiple animals simultaneously.

More specifically, a PET image is a representation of a distribution of radionuclide based on the tomographic reconstruction from lines of response deduced from the detection of coincident photon events due to the annihilation of positrons with electrons. This representation is distorted in part by the count-limited nature of the data acquisition (noise) and by resolution-degradation effects; such as annihilation photon non-collinearity, detector response function and positron range. In the case of I-124, positron range plays a significant role in resolution degradation.

The image ρ′ (ignoring the contribution of noise, scatter, random and prompt gamma events) corresponding to a three-dimensional (3D) distribution of PET radionuclide ρ can be represented by:

ρ′=S*R_{I-124}*G_{F-18}*ρ (3)

where G_{F-18} is a 3D Gaussian kernel representing the image blur due to the F-18's annihilation photon non-collinearity, detector response function and positron range. R_{I-124}is a 3D Gaussian kernel representing the additional degradation in resolution due to the positron range of I-124, S is the filter applied to the reconstructed image and voxel discretization (binning effect), and * is the convolution operator. The image blur introduced by the F-18 positron range is negligible compared with the intrinsic resolution of the PET scanner, therefore the kernel G_{F-18} can be taken as representative of the kernel due to the intrinsic resolution of the PET scanner. Alternatively one could represent the resolution degradation due to the system and the positron range as a single convolution using data measured from a single point-spread function measurement for I-124 so

ρ′=S*G_{I-124}*ρ (4)

Assuming a homogeneous, spherical activity distribution of known volume V surrounded by a background of known constant activity concentration, it is possible to calculate recovery coefficients R, in a manner similar to that disclosed in “Analysis of Emission Tomographic Scan Data: Limitations Imposed by Resolution and Background”—Robert M. Kessler, James R. Ellis, Jr., and Murry Eden Jou. Comp. Ass. Tomo 8(3): 514-522. This recovery coefficient is defined as:

$$R = \frac{{ρ}_{max}}{{ρ}_{max}^{′}} \qquad ( 5 )$$

Recovery coefficients were calculated using Equation (4) for simulated spherical activity distributions with volumes, background-to-target ratios and distances from the scanner's z-axis equal to those of the phantom measurements. The simulated distributions were then convolved with Gaussian PSFs according to Equation (3). The measured experimental (exp) maximum activity concentrations ρₘₐₓ^{exp} in the I-124 phantom spheres were multiplied by the appropriate recovery coefficient R to obtain the recovered activity concentration ρ_{R}. The recovered activity concentrations were then compared to the true activity concentration in the phantom spheres ρₜᵣᵤₑ=0.74±0.05 μCi/cc.

The overall recovered concentration bias was estimated as the mean percent difference for all recovered activity concentrations measured. In the following equations d is the distance of the target from the z-axis of the scanner, V is the volume of the target, b is the target-to-backround ratio. More specifically:

$$\begin{matrix}\% \overline{Δ} = \frac{1}{{N}_{d} {N}_{V} {N}_{b}} {\sum }_{d} {\sum }_{V} {\sum }_{b} \% Δ ( d , V , b ) & ( 6 ) \\ where \\ \% Δ ( d , V , b ) = 100 \frac{{ρ}_{R} ( d , V , b ) - {ρ}_{true}}{{ρ}_{true}} & ( 7 )\end{matrix}$$

The standard deviation from the percent differences from each individual sphere measurement was given by:

$$\% {Δ}_{s} = {( \frac{1}{{N}_{d} {N}_{V} {N}_{b} - 1} \cdot {\sum }_{d} {\sum }_{V} {\sum }_{b} {( \% Δ ( d , V , b ) - \% \overline{Δ} )}^{2} )}^{\frac{1}{2}} \qquad ( 8 )$$

### EXAMPLE

A six-week-old inbred C.B17/lcr-scid mouse was obtained from the Fox Chase Cancer Center Laboratory Animal Facility. Approximately two months prior to the initiation of the biodistribution study, 3×10⁶ SK-OV-3 cells were injected sub-cutaneously into the thigh of the mouse. The dissected tumor mass shortly after the time of imaging was 0.67±0.07 g.

Thirty-seven microcuries of I-124 labeled antibody were administered by tail vein injection. The total injected dose was determined by counting the mouse on a Capintec Captus 600 thyroid scanner (Ramsey, N.J., USA). Blood sample collection and whole body counts were performed immediately after injection just prior to euthanization. The mouse was anesthetized with Avertin (Winthrop Labs) and three sets of 10 minute images were acquired on a Discovery LS PET/CT system. Images were reconstructed and tumor activity concentration quantitated using the same protocol as for the phantom acquisitions.

In accordance with a third aspect of the invention a distribution of PET radionuclide ρ is assumed to exist within a certain material medium m which contains electrons on which positrons may annihilate to produce a gamma emission and thus a detectable signal. Positrons will annihilate within a certain distance from their radionuclide of origin. This distance depends in a stochastic way on the energy of emission of the positron and the electron density of the surrounding medium. The resulting distribution of annihilation events can be modeled by convolution with a kernel K_{β}_{⁺},

ρ_{β}_{⁺}=K_{β}_{⁺}*ρ (9)

In the present case we assume that the medium m consists of a homogeneous region of relatively high electron density (water-like) and a homogeneous region of relatively low electron density (air-like). A mask M is created which assigns values of 1 to the medium regions with high electron density and 0 to the regions of low electron density. The mask is used as a factor on the annihilation distribution ρ_{β}_{⁺} so that events that fall in the low-density region are suppressed, yielding the corrected annihilation distribution

ρ_{β}_{⁺}′=M·K_{β}_{⁺}*ρ (10)

that is further convolved by the system point-spread function K_{sys}, resulting in the simulation of the estimated activity distribution that would be measured by a PET system

ρ′=K_{sys}MK_{β}_{⁺}*ρ (11)

This estimated activity distribution can then be used to calculate activity concentration recovery coefficients in vivo in the manner described above. In addition, the true activity of assayed tissue may be corrected by dividing it by the recovery coefficient given by:

$${R}_{A} = \frac{\sum {ρ}_{{β}^{+}}^{′}}{\sum ρ} \qquad ( 12 )$$

Herein the numerator represents the summation of all annihilation events produced through the volume of the target object and surrounding material, and the denominator represents the summation of all disintegration events throughout the volume of the target. This calculation is based on the volume of the target tissue (which can be obtained by x-ray CT, for instance) and a priori knowledge of the material surrounding the tissue sample.

A further refinement comprises the empirical (measured) recovery coefficients Rₑ for distributions that are fully embedded in the attenuation medium. Then the calculated (by the previously described convolution procedure or some other method) recovery coefficients for fully embedded Rₛ^{c} and partly embedded Rₚ^{c} target distribution are calculated and their ratio obtained

$$Δ = \frac{{R}_{p}^{c}}{{R}_{s}^{c}} \qquad ( 13 )$$

and used as a correction factor on the empirical recovery coefficients in order to obtain a semi-empirical recovery coefficient for the semi-embedded target distribution

R^{se}=Δ·Rₑ (14).

This procedure has the advantage of applying a perturbative correction to an empirical (and presumably accurate) recovery coefficient value, and thus introducing less sensitivity to the model used for the correction.

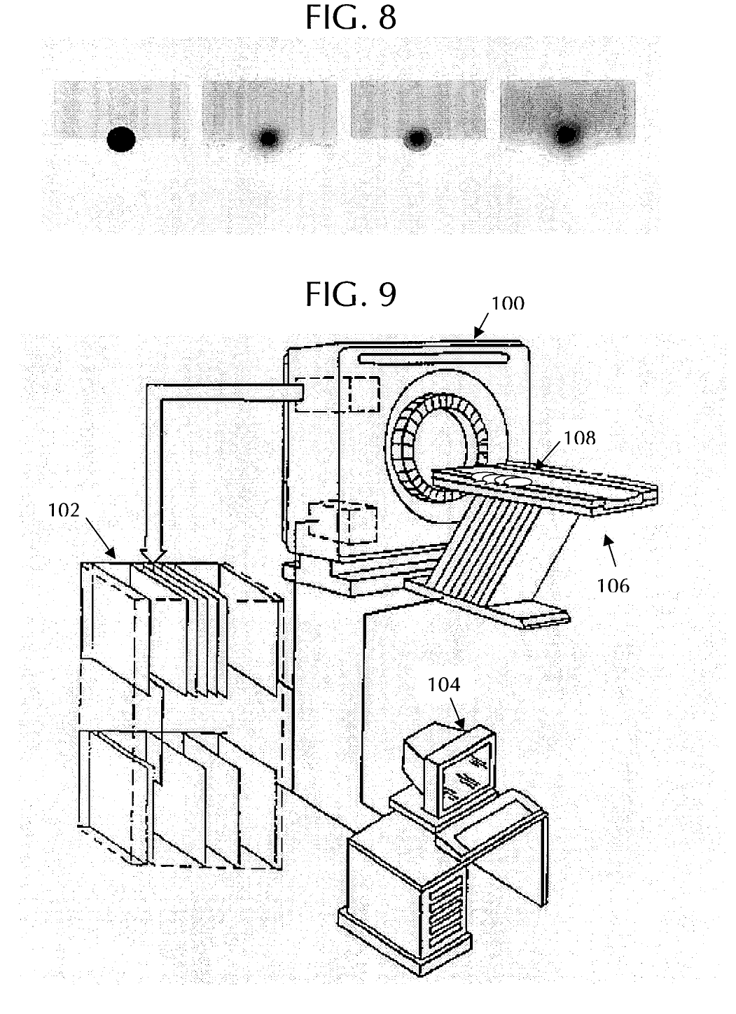

FIG. 8 shows an example of a spherical distribution of activity embedded in a background of activity which occupies only part of the space surrounding the spherical distribution. A prototype code written in Matlab was used in the generation of activity concentration recovery coefficients R and activity recovery coefficients R_{A}. These coefficients have been compared to experimental PET and scintillator counter data, and found to be in excellent agreement.

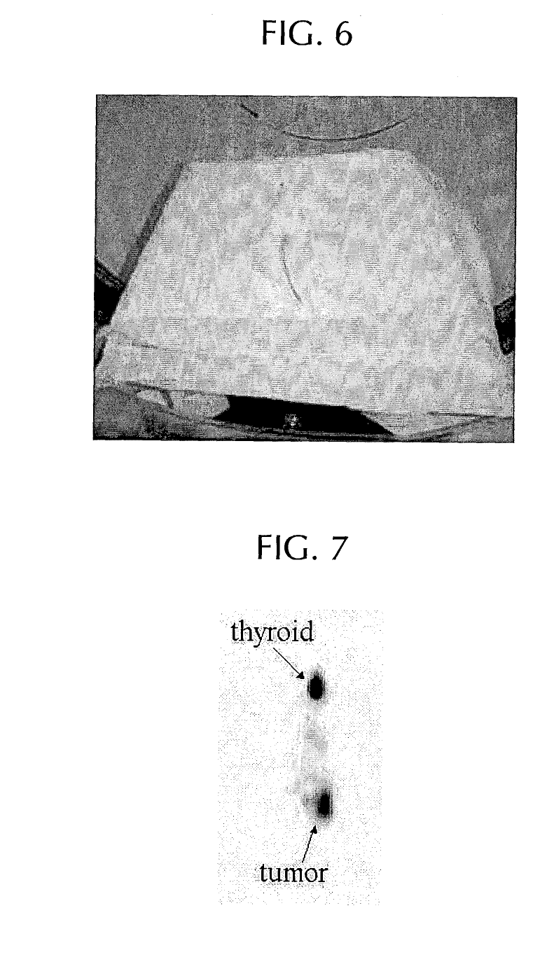

FIG. 9 schematically depicts a PET scanner 100 of the nature which can be used in accordance with the embodiments of the invention. This arrangement includes a memory (including look-up table data) 102 and a control console 104. The movable bed 106 is used, in accordance with the embodiments of the invention to support a plurality of small animals (such as the mouse shown in FIG. 6), herein schematically depicted at 108 and thus allow the simultaneous scanning of multiple animals and speeds up the examination throughput.

It should be further noted that it is within the aspects of the invention to calculate corrected activity concentrations of target tissues from a PET image by multiplying together the selected recovery coefficient from a look-up table, and the maximum activity concentration of the target tissue as determined from the uncorrected PET image.

In this instance the selected recovery coefficient is obtained by look up based on predetermined parameters including shape, size, and position respectively determined by a predetermined process which includes X-ray scanning, CT scanning, and using PET reconstruction algorithm.

It will be understood that while the invention has been described with reference to only a limited number of embodiments, the invention, is limited only by the appended claims and various changes and modifications can be made without departing from the scope thereof. That is to say, the processes which are described above can be applied to research related to the application of any suitable labeling tracer and used to assay the concentration of the tracer within a small volume such as the organs/tissues of mice or other small animals. The tracers which have been described are merely exemplary of those to which the present invention is applicable.

What is claimed is:

1. A method comprising image point tracer evaluation using PET (positron emission tomography) in small animals in a large bore PET scanner.

2. A method of tracer evaluation using PET (positron emission tomography), comprising:
introducing the tracer into a small animal;
scanning the animal in a large-bore PET scanner; and
estimating a concentration distribution of the tracer in a predetermined portion of the animal.

3. A method as set forth in claim 2, further comprising:
placing a phantom containing inserts which are of known size and containing known quantities of tracer and which are immersed in an attenuating media, in the bore of a large bore PET scanner;
scanning the phantom to provide empirical data; and
developing at least one correction coefficient based on the empirical data.

4. A method of evaluating tracers in a small animals comprising the steps of:
introducing the tracer into a plurality of small animals;
simultaneously scanning the plurality of small animals in a large-bore PET scanner; and
estimating a concentration distribution of tracer in the plurality of small animals.

5. A method as set forth in claim 4, further comprising the steps of:
waiting a predetermined length of time after a first scanning of the small animals;
repeating the scanning of the small animals in the large-bore PET scanner; and
estimating a concentration distribution of tracer in the plurality of small animals.

6. A method as set forth in claim 4, further comprising:
placing a phantom containing inserts which are of known size and containing known quantities of tracer and which are immersed in an attenuating media, in the bore of a large bore PET scanner;
scanning the phantom to provide empirical data; and
developing at least one correction coefficient based on the empirical data.

7. A method as set forth in claim 6, further comprising: storing N-dimensional recovery coefficients in a look-up-table, wherein the N-dimensions comprise N respective parameters comprising at least one of geometry, source-to-background ratio, radio-isotope, and reconstruction method.

8. A method as set forth in claim 7, further comprising:
calculating corrected activity concentrations of target tissues from a PET image by multiplying together the selected recovery coefficient from a look-up table, and the maximum activity concentration of the target tissue as determined from the uncorrected PET image.

9. A method as set forth in claim 8, wherein the selected recovery coefficient is obtained by look up based on predetermined parameters including shape, size, and position respectively determined by a predetermined process.

10. A method as set forth in claim 9, wherein the predetermined process includes X-ray scanning, CT scanning, and using PET reconstruction algorithm.

11. A method as set forth in claim 7, further comprising the steps of avoiding repetitive calculation of recovery coefficients by:
pre-calculating a plurality of recovery coefficients for a range of different parameters and storing the plurality of recovery coefficients in a look up table; and
obtaining the recovery coefficients from the look up table based by interpolation based on the predetermined parameters.

12. A non-invasive method of measuring pharmacological agent distribution at different time points, comprising:
labeling the pharmacological agent with a tracer;
dosing a plurality of small animals with the labeled pharmacological agent;
placing the small animals in a large-bore PET (positron emission tomography) scanner;
scanning the small animals; and
determining a concentration distribution of the tracer in the small animals.

13. A non-invasive method comprising: quantitative imaging in a large-bore scanner for PET (positron emission tomography) tracer/drug assessment.

14. A non-invasive method as set forth in claim 13, wherein the quantitative imaging comprises quantitative imaging of multiple small animals using the large-bore scanner.

15. A non-invasive method as set forth in claim 14, further comprising computing recovery coefficients to apply to small-animal PET data derived from the quantitative imaging of the multiple small animals.

16. A non-invasive method as set forth in claim 15, further comprising: storing N-dimensional recovery coefficients in a look-up-table, wherein the N-dimensions comprise N respective parameters comprising at least one of geometry, source-to-background ratio, radio-isotope, and reconstruction method.

17. A non-invasive method as set forth in claim 13, further comprising using a combination of empirical measurements and convolution kernels to calculate event loss due to positron range.

18. A method of operating a PET (positron emission tomography) scanner to account for loss of events and quantitative inaccuracies in PET imaging, comprising:
modeling a distribution of positron-electron annihilation events by:
convolving a first distribution of positron-emitting radionuclides using a first predetermined kernel to produced a second distribution result;
masking the second distribution result with a three-dimensional map of a physical medium where the distribution of radionuclides is located so as to suppress positron-electron annihilation events from a low-density medium and to produce a third distribution result; and
convolving the third distribution result with a kernel representative of the measurement system to produce a fourth distribution result which takes into account the loss of positron-electron annihilation events.

19. A method as set forth in claim 18, further comprising obtaining resolution recovery coefficients by comparing the first distribution of radionuclide, with the third distribution result.

20. A method as set forth in claim 18, further comprising estimating a loss of positron-electron annihilation events due to a finite size of the physical medium and correcting measured activities using the estimated loss.
